(19) United States
(12) Patent Application Publication
(10) Pub. No.: US 2013/0345641 A1
(43) Pub. Date: Dec. 26, 2013

(54) Coding System for a Drug Delivery Device and Drug Delivery System

(71) Applicants: Zdenek Cerman, Frankfurt am Main (DE); Timothy Lewis Sharpe, Warwickshire (GB); Christopher Nigel Langley, Warwickshire (GB); Richard James Vincent Avery, Gloucestershire (GB); Joseph Butler, Warwickshire (GB); Michael Heald, Maidenhead (GB)

(72) Inventors: Zdenek Cerman, Frankfurt am Main (DE); Timothy Lewis Sharpe, Warwickshire (GB); Christopher Nigel Langley, Warwickshire (GB); Richard James Vincent Avery, Gloucestershire (GB); Joseph Butler, Warwickshire (GB); Michael Heald, Maidenhead (GB)

(73) Assignee: SANOFI-AVENTIS DEUTSCHLAND GMBH, Frankfurt am Main (DE)

(21) Appl. No.: 13/814,697

(22) PCT Filed: Aug. 11, 2011
(86) PCT No.: PCT/EP11/63844

§ 371 Date: Sep. 11, 2013

(30) Foreign Application Priority Data
Aug. 13, 2010 (US) 61373376
Oct. 26, 2010 (EP) 10188850.1

(51) Int. Cl. A61M 5/315

(52) U.S. Cl. CPC A61M 5/31566
USPC 604/189

(57) ABSTRACT

A system for reducing the risk of an incorrect selection or dispensing of a drug dose for use with a cartridge and a cartridge holder so as to prevent unwanted reservoir cross use. The coding system for use with the drug delivery device includes a first partial code feature and a second partial code feature. The first partial code feature and the second partial code feature cooperate to supply a combined visual indicator when the cartridge is assembled to the holder for indicating that a correct cartridge has been assembled to the holder.

### CROSS REFERENCE TO RELATED APPLICATIONS

The present application is a U.S. National Phase Application pursuant to 35 U.S.C. §371 of International Application No. PCT/EP2011/063844 filed Aug. 11, 2011, which claims priority to U.S. Patent Application No. 61/373,376 filed Aug. 13, 2010 and European Patent Application No. 10188850.1 filed Oct. 26, 2010. The entire disclosure contents of these applications are herewith incorporated by reference into the present application.

### FIELD OF INVENTION

The present patent application is generally directed to a coding system for components of a drug delivery device, such as reservoirs and reservoir holders, particularly reservoirs containing a medicament. More particularly, the present application is generally directed to a coding system for a drug delivery device which may prevent unwanted reservoir cross use. As just one example, such medicament reservoirs may comprise an ampoule, a cartridge, a vial, or a pouch, and may be used with a medical delivery device. Exemplary reservoir holders form part of medical delivery devices such as but not limited to syringes, pen type injection syringes, pumps, inhalers, or other similar injection or infusing devices that require at least one reservoir containing at least one medicament.

### BACKGROUND

Medicament reservoirs such as ampoules, cartridges, or vials are generally known. Such reservoirs are especially used for medicaments that may be self administered by a patient. For example, with respect to insulin, a patient suffering from diabetes may require a certain amount of insulin to either be injected via a pen type injection syringe or infused via a pump. With respect to certain known reusable pen type drug delivery devices, a patient loads a cartridge containing the insulin into a proximal end of a cartridge holder. After the cartridge has been correctly loaded, the user may then be called upon to select a dose of medicament. Multiple doses may be delivered from the cartridge. Where the drug delivery device comprises a reusable device, once the cartridge is empty, the cartridge holder is disconnected from the drug delivery device and the empty cartridge is removed and replaced with a new cartridge. Most suppliers of such cartridges recommend that the user dispose of the empty cartridges properly. Where the drug delivery device comprises a disposable device, once the cartridge is empty, the user is recommended to dispose of the entire device.

Such known self administration systems requiring the removal and reloading of empty cartridges have certain limitations. For example, in certain generally known systems, a user simply loads a new cartridge into the delivery system without the drug delivery device or without the cartridge having a mechanism of preventing use of an incorrect cartridge. That is, the drug delivery device does not have a system or mechanism for determining if the medicament contained in the cartridge is indeed the correct type of medicament to be administered by the patient. Alternatively, certain known drug delivery devices do not present a mechanism for determining if the correct type of medicament within the cartridge should be used with that particular drug delivery system. This potential problem could be exacerbated given that certain elderly patients, such as those suffering from diabetes, may have limited manual dexterity and/or visual acuity. Identifying an incorrect medicament is quite important, since the administration of a potentially incorrect dose of a medicament such as a short acting insulin in lieu of a long insulin could result in injury or even death.

Some drug delivery devices or systems may use a color coding scheme to assist a user or care giver in selecting the correct cartridge to be used with a drug delivery device. However, such color coding schemes pose challenges to certain users, especially those users suffering from poor eyesight or color blindness: a situation that can be quite prevalent in patients suffering from diabetes.

Another concern that may arise with such disposable cartridges is that these cartridges are manufactured in essentially standard sizes and must comply with certain recognized local and international standards. Consequently, such cartridges are typically supplied in standard sized cartridges (e.g., 3 ml cartridges). Therefore, there may be a variety of cartridges supplied by a number of different suppliers and containing a different medicament but they may fit a single drug delivery device. As just one example, a first cartridge containing a first medicament from a first supplier may fit a medical delivery device provided by a second supplier. As such, a user might be able to load and then dispense an incorrect medicament (such as a rapid or basal type of insulin) into a reusable drug delivery device without being aware that the medical delivery device was perhaps neither designed nor intended to be used with such a cartridge.

### SUMMARY

As such, there is a growing desire from users, health care providers, care givers, regulatory entities drug manufacturers, and medical device suppliers to reduce the potential risk of a user loading an incorrect drug type into a drug delivery device. There is also, therefore, a desire to reduce the risk of dispensing an incorrect medicament (or the wrong concentration of the medicament) from such a drug delivery device.

There is also a general need to provide a dedicated cartridge that is difficult to tamper with so that the cartridge may not be compromised in that the cartridge can be used with an unauthorized drug or drug delivery device. Because such cartridges may be difficult to tamper with, they may also reduce the risk of counterfeiting: i.e., making it more difficult for counterfeiters to provide unregulated counterfeit medicament carrying products.

It is an object of the invention to simplify a correct use of drug reservoirs or cartridges in conjunction with a drug delivery device and to help to avoid an incorrect selection of a drug reservoir.

This object is achieved with the coding system according to claim 1 and with the drug delivery system according to claim 15, respectively. Further embodiments derive from the dependent claims.

According to an exemplary arrangement, a combination coding mechanism for use with a drug delivery device is disclosed. In a most general form, the invention includes a combination coding mechanism that includes a first coding feature that cooperates with a second coding feature to provide a visual indication that a correct cartridge is being used with a correct cartridge holder, for example.

According to an example arrangement, a coding system for use with a drug delivery system includes a first partial code feature disposed on a first drug delivery system feature and a second partial code feature disposed on a second drug delivery system feature. The first drug delivery device system is connectable to the second drug delivery system feature. Further, the first partial code feature and the second partial code feature cooperate to supply a combined information code when the first drug delivery system feature is connected to the second drug delivery system feature.

According to a preferred embodiment, the first drug delivery system feature is some kind of standard feature which in principal fits with a variety of different types of second drug delivery system features. The different types may all have a similar size and shape and therefore at least being suggestive of all fitting with the first drug delivery system feature. The type of the second drug delivery system feature may be defined by the supplier providing the feature such that features of supplier A belong to type A features and features from supplier B belong to type B features. Furthermore, the type of the second drug delivery system feature may be defined by different medicaments to which the second feature is dedicated or by different concentrations of the same medicament to which the second feature is dedicated. The different types of second drug delivery system features may include at least on correct type of feature which is dedicated to cooperate with the first drug delivery system feature and at least one incorrect type which is not dedicated to cooperate with the first drug delivery system feature. The different types of second drug delivery system features may have different second partial coding features disposed thereon. These different coding features are chosen such that only the coding feature of the correct type of second drug delivery system can form the combined information code in combination with the first partial code feature. Generally, the combined information code is only supplied in case that the correct second drug delivery system feature is connected to the first drug delivery system feature. In case that no further mechanical coding of the first and second drug delivery system feature is present, the different types of second drug delivery system features all fit with the first drug delivery system feature, e.g. can be mounted to the first drug delivery system feature in fact and not only in principle. “Fitting in principle” can mean that in view of shape an size and in view of the mounting features two parts fit. It can further mean that a significant share of the mounting movement between these two parts can be performed before a coding feature hinders further mounting movement.

For example, the first drug delivery system feature may be a dose setting mechanism dedicated for the administration of a certain fixed dose of a certain type of insulin. The dose setting mechanism is to be mounted to a cartridge holder dedicated for holding a cartridge filled with a certain type of medicament to be administered. The cartridge holder is the second drug delivery system feature in this case, and a variety of different types of cartridge holders exist, all of which apparently fitting with the dose setting mechanism. The different types of cartridge holders are dedicated to different types of medicament or medicament concentration. However, the combined information code is only supplied in case that the cartridge holder dedicated to the correct medicament or concentration of medicament, respectively is connected to the dose setting mechanism.

According to another example arrangement, a coding system for use with a drug delivery device that includes a holder and a reservoir is provided. The reservoir may be chosen out of a variety of different types of reservoirs being sized and shaped to be received within the holder. The variety of reservoirs may include at least one incorrect reservoir which is not dedicated for use with the holder. The coding system includes a first partial code feature and a second partial code feature. The first partial code feature is disposed on the reservoir and the second partial code feature is disposed on the reservoir holder. The first partial code feature and the second partial code feature cooperate to provide a combined visual indicator when the reservoir is assembled to the holder for indicating that a correct reservoir has been assembled to the holder. Additional mechanical coding may be used to physically prevent connection of a reservoir to an incorrect holder, and/or to prevent assembly of a holder to an incorrect dose setting mechanism.

According to a preferred embodiment, the cartridge holder is some kind of standard cartridge holder which fits with a variety of different types of cartridges. The type of cartridge may be defined by the supplier providing the cartridge such that cartridges of supplier A belong to type A cartridges and cartridges from supplier B belong to type B cartridges. Both types fit with the cartridge holder. Furthermore, the type of cartridge may be defined by different medicaments contained in the cartridge or by the concentrations of the same medicament contained. In this regard the combination coding mechanism indicates that the correct type of cartridge is loaded in the cartridge holder. For example, the cartridge holder designated for the administration of insulin is loaded with an insulin cartridge and not with a cartridge containing a different medicament.

In one aspect, a coding system for use with a drug delivery device comprising a holder and a reservoir sized and shaped to be received within the holder comprises a first partial code feature; and a second partial code feature, wherein the first partial code feature and the second partial code feature cooperate to supply a combined code feature when the reservoir is assembled to the holder for indicating that a correct reservoir has been assembled to the holder.

In an example embodiment, the combined code feature comprises a visual indicator.

In a further example embodiment, the visual indicator is one or more of a drug name, alphanumeric character, image, icon, symbol, design, trademark, word, phrase, number, or indicia.

In a further example embodiment, the combined code feature comprises a three-dimensional Braille code.

In a further example embodiment, the combined code feature comprises a bar code.

In a further example embodiment, the reservoir includes one of an ampoule, a cartridge, a vial, or a pouch.

In a further example embodiment, the first partial code feature is disposed on an outside surface of the reservoir.

In a further example embodiment, the first partial code feature is disposed on an inside surface of the reservoir.

In a further example embodiment, the first partial code feature is embedded within the material forming the reservoir.

In a further example embodiment, the first partial code feature is provided to the reservoir on a label.

In a further example embodiment, the first partial code feature is provided to the reservoir on an adapter.

In a further example embodiment, the first partial code feature includes one or more shaped areas.

In a further example embodiment, the shaped areas have a predetermined shape.

In a further example embodiment, the shaped areas are a predetermined shape formed of one or more color.

In a further example embodiment, the second partial code feature is one or more window formed through the holder, such that the one or more shaped areas can be viewed.

In a further example embodiment, the one or more window corresponds in shape and size to the one or more shaped areas.

In a further example embodiment, each of the one or more shaped areas is an elongate shape.

In a further example embodiment, the one or more shaped areas includes a plurality of elongate shapes, which may particularly be formed of the color green, for instance, and the second partial code feature includes a number of windows formed through the holder corresponding in number, size and shape to the plurality of elongate shapes.

In a further example embodiment, the coding system further comprises a third partial code feature, wherein the first partial code feature, the second partial code feature and the third partial code feature cooperate to supply a combined code feature when the reservoir is assembled to the holder for indicating that a correct reservoir has been assembled to the holder.

In a further example embodiment, the coding system further comprises an alignment feature, wherein the alignment feature aids the first partial code feature and the second partial code feature to cooperate so as to supply a combined code feature when the reservoir is assembled to the holder for indicating that a correct reservoir has been assembled to the holder.

In a further example embodiment, the coding system further comprises a protrusion, wherein the protrusion is configured to mesh with either the reservoir holder or the reservoir so as to align the first and second partial codes.

In a further example embodiment, the holder is mechanically coded to the dose setting mechanism.

In one aspect, a coding system for use with a drug delivery system comprises a first partial code feature disposed on a first drug delivery system feature and a second partial code feature disposed on a second drug delivery system feature, wherein the first drug delivery device system is connectable to the second drug delivery system feature, wherein the first partial code feature and the second partial code feature cooperate to supply a combined information code when the first drug delivery system feature is connected to the second drug delivery system feature.

In a further example embodiment, the first drug delivery system feature is a holder, and the second drug delivery system feature is a dose setting mechanism housing.

In a further example embodiment, the first drug delivery system feature is a drug reservoir, and the second drug delivery system feature is a reservoir holder.

In a further example embodiment, the first drug delivery system feature is a cap, and the second drug delivery system feature is an injection device.

In a further example embodiment, the first drug delivery system feature is a cap, and the second drug delivery system feature is a cartridge holder.

In a further example embodiment, the first drug delivery system feature is a cartridge holder, and the second drug delivery system feature is a cartridge.

In a further example embodiment, the first drug delivery system feature is a drug reservoir, and the second drug delivery system feature is a dose setting mechanism housing.

In a further example embodiment, the combined code feature comprises a visual indicator.

In a further example embodiment, the visual indicator is one or more of a drug name, alphanumeric character, image, icon, symbol, design, trademark, word, phrase, number, or indicia.

In a further example embodiment, the combined code feature comprises a three-dimensional Braille code.

In a further example embodiment, the combined code feature comprises a bar code.

In a further example embodiment, a coding system for use with a drug delivery device comprises a drug reservoir comprising a three-dimensional coding feature disposed on an external portion of the outer body and a corresponding holder for receiving the drug reservoir, the holder comprising a corresponding coding feature for receiving the three-dimensional coding feature.

In a further example embodiment, the corresponding coding feature is a negative impression in an inner surface of the holder, wherein the negative impression is substantially the same shape as the three-dimensional coding feature.

In a further example embodiment, the corresponding coding feature is a cut out that is substantially the same shape as the three-dimensional coding feature.

In a further example embodiment, the drug reservoir further comprises a connection feature for attaching a dispense interface, and, when a user attempts to attach the dispense interface to the drug reservoir, the drug reservoir is prevented from rotating relative to the holder.

In a further example embodiment, a drug delivery system comprises a drug delivery device comprising a dose setting mechanism and a cartridge holder removably coupled to the dose setting mechanism, a cartridge sized and shaped to be contained within the cartridge holder and operably responsive to the dose setting mechanism, and a coding system for use with the drug delivery device including a first partial code feature and a second partial code feature, wherein the first partial code feature and the second partial code feature cooperate to supply a combined visual indicator when the cartridge is assembled to the cartridge holder for ensuring that a correct cartridge has been assembled to the holder.

In a further example embodiment, the drug delivery system comprises a reusable drug delivery system.

In a further example embodiment, the drug delivery system comprises a non-reusable drug delivery system.

The term “drug”, as used herein, preferably means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyhepta-decanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

These as well as other advantages of various aspects of the present invention will become apparent to those of ordinary skill in the art by reading the following detailed description, with appropriate reference to the accompanying drawings.

### BRIEF DESCRIPTION OF THE DRAWINGS

Exemplary embodiments are described herein with reference to the drawings, and wherein similar reference characters denote similar elements throughout the several views, in which:

FIG. 1a illustrates an exemplary pen type drug delivery device;

FIG. 1b illustrates a cartridge that may be loaded into a cartridge holder of the pen type drug delivery device illustrated in FIG. 1;

FIGS. 2a, 2b and 2c illustrate an example of a combined coding system for use with a cartridge that may be used with a pen type drug delivery device, such as the drug delivery device shown in FIG. 1a;

FIGS. 3a, 3b and 3c illustrate an alternate example combined coding system;

FIGS. 4a, 4b and 4c illustrate yet another example combined coding system;

FIGS. 5a and 5b illustrate an example coding system for a drug delivery device;

FIGS. 6a, 6b, and 6c illustrate example coding features of an example coding system where the visual indicator of the coding system is a drug name;

FIGS. 7a, 7b, and 7c illustrate example coding features of an example coding system where the visual indicator of the coding system is a bar code;

FIGS. 8a and 8b illustrate yet another example coding system for a drug delivery device;

FIGS. 9a and 9b illustrate yet another example coding system for a drug delivery device, where the code comprises a Braille message;

FIG. 10 illustrates an example coded drug cartridge having a three-dimensional code feature;

FIGS. 11a, 11b and 11c illustrate yet another example combined coding system;

FIG. 12 illustrates an example system for a drug delivery device for reading a code and reacting to the identified code; and

FIG. 13 illustrates an example pouch that may be coded and loaded into a suitable dispensing device.

### DETAILED DESCRIPTION

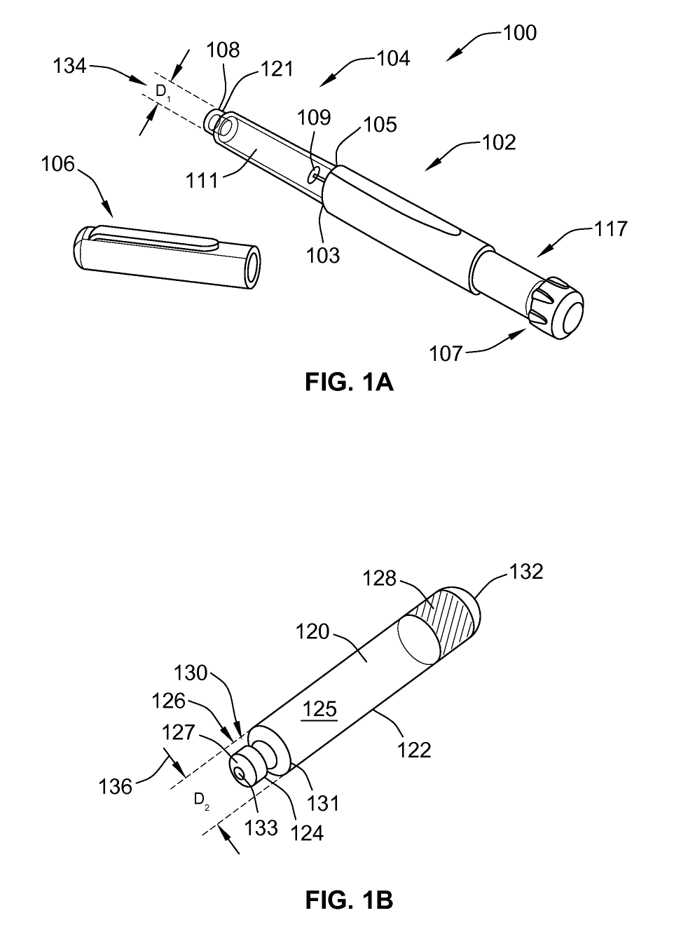

FIG. 1a illustrates a drug delivery device 100 in the form of a pen type syringe. This drug delivery device 100 comprises a dose setting mechanism 102, a cartridge holder 104, and a removable cap 106. A proximal end 105 of the cartridge holder 104 and a distal end 103 of the dose setting mechanism 102 are removably secured together. The pen type syringe may comprise a re-usable or a disposable pen type syringe. Where the syringe comprises a reusable device, the cartridge holder 104 and the dose setting mechanism are removably coupled together. In a disposable device, they are permanently coupled together. In FIG. 1, the dose setting mechanism 102 comprises a piston rod 109, such as a threaded piston rod that rotates when a dose is injected.

To inject a previously set dose, a double ended needle assembly (not shown) is attached to a distal end 108 of the cartridge holder. Preferably, the distal end of the holder comprises a thread 121 (or other suitable connecting mechanism such as a snap lock, snap fit, form fit, or bayonet lock mechanism) so that the needle assembly may be removably attached to the distal end of the holder. When the drug delivery device is not in use, the removable cap 106 can be releasably retained over the cartridge holder 104.

An inner cartridge cavity 111 defined by the cartridge holder 104 is dimensioned and configured to securely receive and retain the cartridge 120. FIG. 1b illustrates a perspective view of the cartridge 120 that may be used with the drug delivery device illustrated in FIG. 1a. The cartridge 120 includes a generally tubular barrel 122 extending from a distal end 130 to a proximal end 132. The distal end 130 is defined by an inwardly converging shoulder 131.

At the distal end 130, the cartridge 120 includes a smaller diameter neck 126 and this neck projects distally from the shoulder 131 of the barrel 122. Preferably, this smaller diameter neck 126 is provided with a large diameter annular bead 133 and this bead extends circumferentially thereabout at the extreme distal end of the neck 126. A pierceable seal or septum 127 is securely mounted across the open distal end defined by the neck. The seal 127 may be held in place by a metallic sleeve or ferrule 124. This ferrule 124 may be crimped around the circumferential bead at the distal end of the neck. The medicament 125 is pre-filled into the cartridge 120 and is retained within the cartridge, in part, by the pierceable seal 127, the metallic sleeve 124, and the stopper 128. The stopper 128 is in sliding fluid-tight engagement with the inner tubular wall of the barrel 122. Axially directed forces acting upon the stopper 128 during dose injection or dose administration urges the medication 125 from the cartridge though a double ended needle mounted onto the distal end 130 of the cartridge holder 104 and into the injection site. Such axial forces may be provided by the piston rod 109.

A portion of the cartridge holder 104 defining the cartridge holder cavity 111 is of substantially uniform diameter represented in FIG. 1a by D1 134. This diameter D1 134 is preferably slightly greater than the diameter D2 136 of the cartridge 120. The interior of the cartridge holder includes an inwardly-extending annual portion or stop that is dimensioned to prevent the cartridge 120 from moving within the cartridge holder 104. In this manner, when the cartridge 120 is loaded into the cavity 111 of the cartridge holder 104 and the cartridge holder 104 is then connected to the dose setting member 102, the cartridge 120 will be securely held within the cartridge cavity. More particularly, the neck 126 and ferrule 124 of the cartridge 120 are inserted in a proximal to distal direction into the open proximal end of the cartridge holder 104 with the ferrule eventually passing entirely into the holder 104. With the holder 104 removably coupled to the dose setting mechanism 102, the proximal end of the cartridge 120 will typically abut a stop provided by the dose setting member 102.

A number of doses of a medicament 125 may be dispensed from the cartridge 120. It will be understood that the cartridge 120 may contain a type of medicament that must be administered often, such as one or more times a day. One such medicament is insulin. A movable piston 128 is retained in a first end or proximal end of the cartridge 120 and receives an axial force created by the piston rod 109 of the dose setting mechanism 102.

The dose setting mechanism 102 comprises a dose setter 117 at the proximal end 107 of the dose setting mechanism. In one preferred arrangement, the dose setter 117 may extend along the entire length of the dose setting mechanism. The dose setter 117 may be rotated by a user so as to set a dose.

To administer a dose that may be set by rotating the dose setter 117, the user attaches the needle assembly comprising a double ended needle on the distal end of the cartridge holder. In this manner, the needle assembly pierces the seal 127 of the cartridge 120 and is therefore in liquid communication with the medicament 125. The user pushes on the dose setter 117 to inject the set dose. The same dose setting and dose administration procedure is followed until the medicament 125 in the cartridge is expended and then a new cartridge must be loaded in the device. To exchange an empty cartridge, the user is called upon to remove the cartridge holder 104 from the dose setting mechanism 102.

A coding system for use with a drug delivery system, such as drug delivery device 100, is provided. Generally, a coding system in accordance with an example of the invention includes a first partial code feature disposed on a first drug delivery system feature and also a second partial code feature disposed on a second drug delivery system feature. The first drug delivery system feature is connectable to the second drug delivery system feature. When the first drug delivery system feature is connected to the second drug delivery system feature, the first partial code feature and the second partial code feature cooperate to supply a combined information code. A user may use the combined information to determine that the first drug delivery system feature is intended for use with the second drug delivery system feature. Example coding systems for a drug delivery system are described below with reference to the figures.

The first and second code features can be arranged between wide varieties of drug delivery system features. For example, the code features can be arranged between: a holder and a dose setting mechanism housing; a drug reservoir and a reservoir holder; a cap and an injection device; a cap and a cartridge holder; a cartridge holder and a cartridge; a drug reservoir and a dose setting mechanism housing. Other combinations of drug delivery system features are possible as well. Further, in given examples, three or more drug delivery system features each having a partial code may be combined to result in a given combined code.

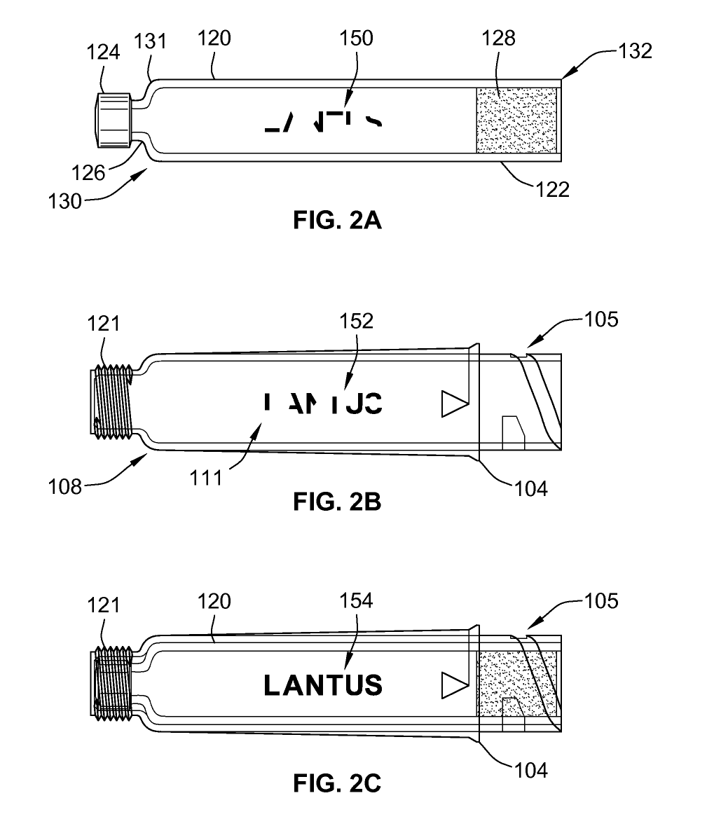

The coding system may be a coding system for use with a drug delivery system like that shown in FIGS. 1a and 1b, where the drug delivery system includes a holder and a reservoir sized and shaped to be received within the holder. An example of such a coding system is shown in FIGS. 2a, 2b, and 2c. The coding system gives the user visual feedback or an indication whether a correct cartridge 120 is inserted into a correct cartridge holder 104.

FIG. 2a shows a reservoir in the form of a cartridge 120 having a distal end 130 opposite a proximal end 132. The cartridge 120 includes a barrel 122, which has a generally hollow, tubular shape, extending between the distal end 130 and the proximal end 132. The barrel 122 includes a shoulder 131, which narrows to a small neck 126 formed adjacent the distal end 130. The distal end 130 is capped with a metallic sleeve or seal 124. Inside the barrel 122 a stopper 128 is positioned near the proximal end 132. Preferably, the cartridge contains a medicament, such as either a long or a short acting insulin.

A first partial code or first partial coding feature 150 is shown disposed on the outside of the barrel 122 between the distal end 130 and the proximal end 132. The first partial code or coding feature 150 is part of a visual indicator or code that is completed or formed when the cartridge 120 is assembled to the cartridge holder 104. Illustrative examples of a first partial code 150 include part of a drug name, alphanumeric character, image, icon, symbol, design, trademark, word, phrase, number, suitable indicia, and the like, or combinations thereof.

In alternate embodiments, the first partial code 150 may be imbedded in the material of the barrel 122 or, alternately disposed on the inside surface of the barrel. A further embodiment comprises providing the cartridge 120 with the first partial code 150 on a label or an interface or connectors attached to the cartridge. Certain labels, interfaces and connectors are well-known in the art, and thus are not described in great detail.

Turning to FIG. 2b, a holder for the reservoir is shown in the form of a cartridge holder 104 including a distal end 108 opposite a proximal end 105. The cartridge holder 104 includes an inner cartridge holder cavity that is shaped and sized to receive the cartridge 120 (see FIG. 2a). The distal end 108 of the cartridge holder 104 includes a thread 121.

A second partial code or second partial coding feature 152 may be disposed on the outside of the cartridge holder 104 somewhere between the cartridge holder distal end 108 and the cartridge holder proximal end 105. In alternate embodiments, the second partial code 152 may be imbedded in the material of the cartridge holder 104 or, alternately disposed on the inside surface of the cartridge holder inner cavity 111.

The second partial code or coding feature 152 is part of a visual indicator or code that is completed or formed cooperatively with the first partial code or feature 150 when the cartridge 120 is assembled to the cartridge holder 104. Illustrative examples of a second partial code 152 include part of a drug name, alphanumeric character, image, icon, symbol, design, trademark, word, phrase, number, suitable indicia, and the like, or combinations thereof.

Referring to FIG. 2c, the cartridge 120 is assembled to the cartridge holder 104 by inserting the cartridge into the inner cavity 111 of the cartridge holder. In so doing, the first partial code 150 is physically and visually superimposed upon the second partial code 152.

It will be understood that the material of at least the cartridge holder 104 permits viewing of the contents positioned inside, i.e., the cartridge, and the first partial code 150 disposed on the cartridge 120 when the cartridge is positioned properly within the cartridge holder. Thus, for example, at least the cartridge holder 104 is transparent or translucent to the extent that permits visual inspection of the interior thereof. In some embodiments, both the cartridge holder 104 and the cartridge 120 are transparent or translucent. This permits viewing of the contents (i.e., a medicament or the like) of the cartridge 120 in addition to the combined first partial code 150 and the second partial code 152. When the first partial code 150 is properly aligned with the second partial code 152, a completed or combined visual indicator 154 is formed by the cooperative visual combination or coordination of the two partial codes.

It will be understood that, in one embodiment, each of the first partial code 150 and the second partial code 152 may not, when viewed individually, convey a complete message to the viewer, but rather, convey that a correct cartridge 120 and cartridge holder 104 have been assembled when a predetermined visual indicator 154 is formed by the combination of the two partial codes. Therefore, the user can confirm that they successfully selected a correct combination of reservoir and holder. However, it should be understood that it is not a requirement that the first partial code 150 and the second partial code 152 fail to convey any meaningful information individually. In an example, a first code may read “Slow-Acting” and the second code may read “Insulin.” In this example, each code individually conveys an at least partially meaningful message; however, combined, the combined code reveals the message of “Slow Acting Insulin,” which may beneficially indicate to the user the type of drug the user is loading into a delivery device.

Referring to the example illustrated in FIGS. 2a, 2b and 2c, the visual indicator is “LANTUS,” which is a trademark for a brand of insulin. LANTUS is composed of letters. In the illustrated example, only a part of the letter “L” is found in the first partial code 150 with the remainder found in the second partial code 152. Similarly, only a part of the letter “A” is found in the first partial code 150 with the remainder found in the second partial code 152. Of course, when the first partial code 150 and the second partial code 152 are visually combined by assembling the cartridge 120 into the cartridge holder 104, the letters are cooperatively completed into a recognizable visual indicator 154. In one arrangement, more than a first and second partial code may be provided. For example, a third partial code may be provided such that first partial code feature, the second partial code feature, and the third partial code feature cooperate so as to supply a combined code feature when the reservoir is assembled to the holder for indicating that a correct reservoir has been assembled to the holder.

The cooperative alignment of the first partial code 150 and the second partial code 152 properly displays the visual indicator 154, and may be assured with the use of structural or visual features on the cartridge, ferrule label, or an interface In one arrangement, an alignment feature is provided wherein the alignment feature aids the first partial code feature and the second partial code feature to cooperate so as to supply a combined code feature when the reservoir is assembled to the holder and helps to indicate that a correct reservoir has been properly assembled to the reservoir or cartridge holder.

Although the first and second partial codes shown in FIG. 2 each contain only a portion of each letter in the word “LANTUS”, this is intended as an example only. In another example, for instance, the first partial code may be “L_N_S” and the second partial code may be “_A_TU_”. When assembled, the completed code would then read “LANTUS”. It should be understood that other combinations of letters are possible as well, in addition to other completed codes. For instance, a drug delivery device may be intended for use with the drug Apidra, and the first partial code may be “A_I_A” and the second partial code may be “_P_DR_”, resulting in a combined code of “APIDRA”.

Given that the combination of a holder and reservoir having a coding system in accordance with the invention will result in an indication that the reservoir and holder are intended for one another, it should be understood that combining a holder and reservoir not intended for one another will not result in such an indication. For instance, if a user combines a cartridge not intended for a given holder, the assembled completed code may be a nonsensical code, thus alerting the user of an incorrect combination. For example, an attempted combination of a Lantus cartridge (e.g., a cartridge having a code “L_N_S”) and an Apidra pen (e.g., a pen having a code “_P_DR_”) would result in a nonsensical code of “LPNDRS”. Thus, a user would be able to determine that the cartridge is not intended for use with the pen.

As noted above, the visual indicator 154 may be text, for example a drug name, alphanumeric characters, images, icons, symbols, designs, trademarks, words, numbers, indicia, and the like, or combinations thereof. Other examples of visual indicators 154 include other visual indicators or signals, as will be explained more fully below.

Additional mechanical coding may be used to physically prevent connection of a reservoir to an incorrect holder, or to prevent assembly of a holder to an incorrect dose setting mechanism. For example, the bayonet used to fasten the holder to the dose setting mechanism may be a different design for each medicament.

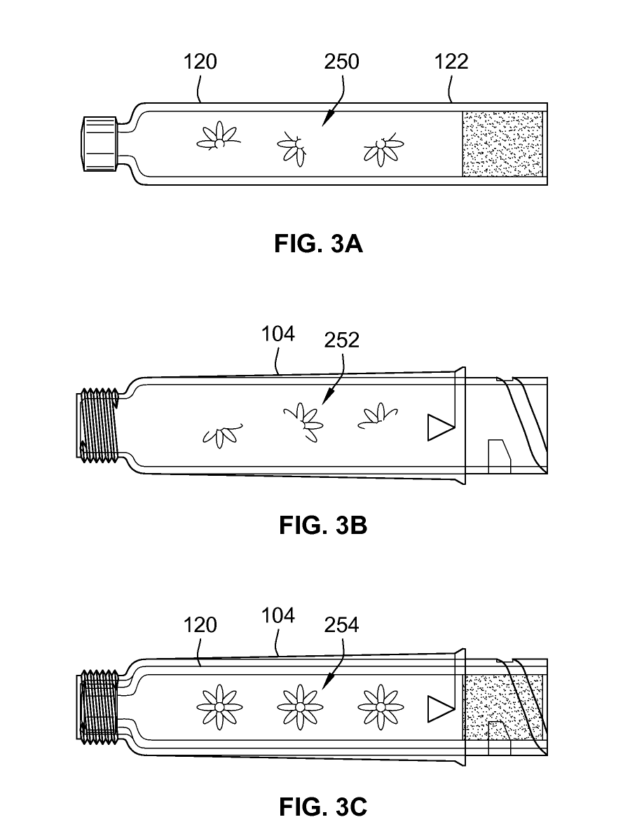

Turning to FIGS. 3a, 3b and 3c, another embodiment of the invention is illustrated with another example of a combined visual indicator 254. In this embodiment, the visual indicator 254 is a combination of a first partial code 250 that is positioned on the barrel 122 of a reservoir in the form of a cartridge 120 and a second partial code 252 that is positioned on a holder in the form of a cartridge holder 104. The visual indicator 254 may, for example, be one or more of an image, symbol, logo, icon or the like and may also include other visual cues, such as words. In the illustrated example, the first partial code 250 is three partial images, each in the form of part of a flower image. The second partial code 252 is three partial images, that combined with the images of the first partial code 250 cooperate to form a complete set of three flowers to form the visual indicator 254.

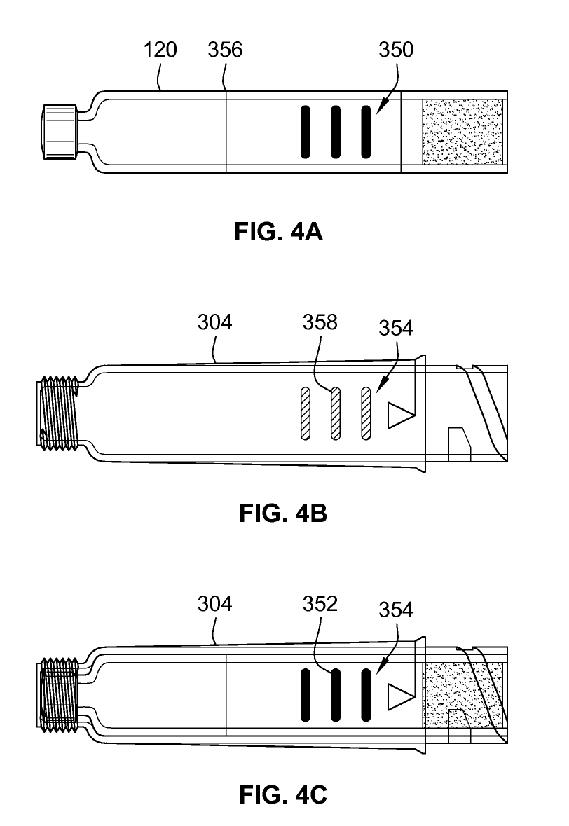

FIGS. 4a, 4b and 4c illustrate yet another embodiment of the invention. Cartridge 120 has a first partial code or cartridge code 350 in the form of one or more predetermined shapes, for example. Specifically, the partial code 350 is in the form of three spaced, elongate shapes and may be formed by areas of color. The shapes may alternatively have no color, and may alternately be provided with a pattern, for example. The partial code 350 may be directly printed or provided on or within the cartridge 120, or may be provided via a label or sleeve 356, for example. It will be understood that the invention contemplates any suitable shape or shapes, images, icons, symbols or the like and any suitable color or pattern forming the first partial code 350. In one further embodiment, the coding may form complete rings around the cartridge, so the cartridge does not have to be rotationally aligned with the holder.

The cartridge holder 304, into which the cartridge 120 is insertable, includes a viewing window 354. The viewing window 354 may include a single opening, or multiple openings as shown. When the correct cartridge 120 is inserted into the correct cartridge holder 304, the first partial code 350 presents a visual indicator 352 to confirm that a correct cartridge 120 has been assembled into a correct cartridge holder 304. In this example, the visual indicator 352 appears as three spaced windows, each having an elongate shape filled with a matching green shape. For instance, as shown in FIG. 4c when the correct cartridge 120 is inserted in the holder 304, the elongate shapes of first partial code are viewable through the viewing windows 354, forming visual indicator 352.

However, if an incorrect cartridge is inserted in the holder 304, a visual indicator such as indicator 352 will not be formed. Each medicament could have a different code, for example with a unique size, color, or position. Referring to FIG. 4b, if a cartridge holder 304 with a viewing window 354 has installed therein an incorrect cartridge, the viewing window will not be aligned with the partial code 350, and an incorrect indicator 358 will be presented to the user. In this embodiment, the negative indicator 358 appears as the lack of a colored shape. It will be understood that any mismatch of code and viewing window, i.e., a lack of the presentation of a predetermined visual indicator will indicate an incorrect cartridge in the cartridge holder.

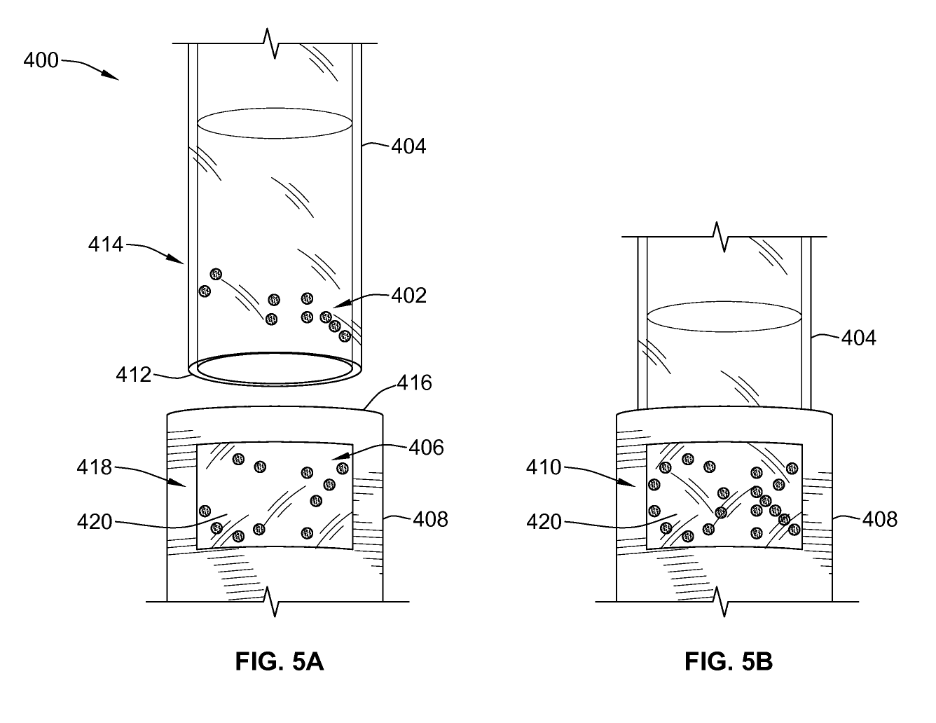

FIGS. 5a and 5b illustrate yet another example coding system for use with a drug delivery system. In particular, FIGS. 5a and 5b illustrate coding system 400 for a drug delivery device. In this example, a first partial code feature 402 is disposed on holder 404, and a second partial code feature 406 is disposed on housing 408. The first partial code feature 402 is located on the distal end 412 of cartridge holder 404 and includes of a collection of dots 414. The second partial code feature 406 is located on proximal end 416 of the housing 408 and includes a second collection of dots 418. The housing 408 also includes a window or lens 420, which allows the code feature 402 to be seen through the housing 408 when the holder 404 is attached to the housing 408. As shown in FIG. 5b, when the holder 404 is connected to the housing 408, the dots 414 and 418 of code fragments 402 and 406 form a combined information code 410. In this example, the combined information code 410 is an “OK” indication, which may beneficially indicate to the user that the holder 404 is intended for use with housing 408. The user may then be confident that the holder and housing connected to one another are intended for use with one another.

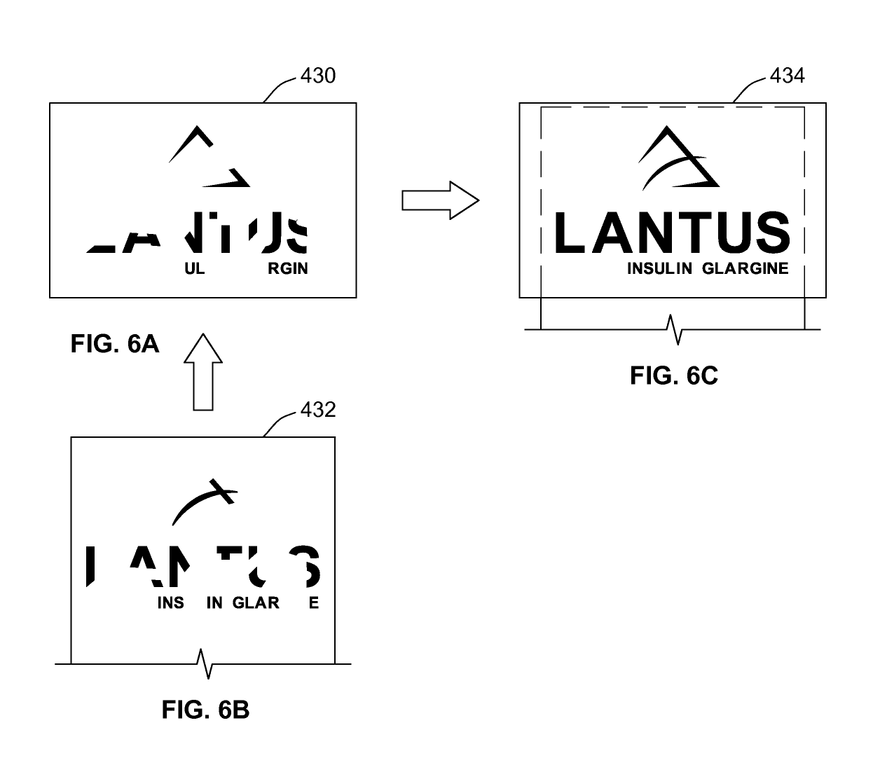

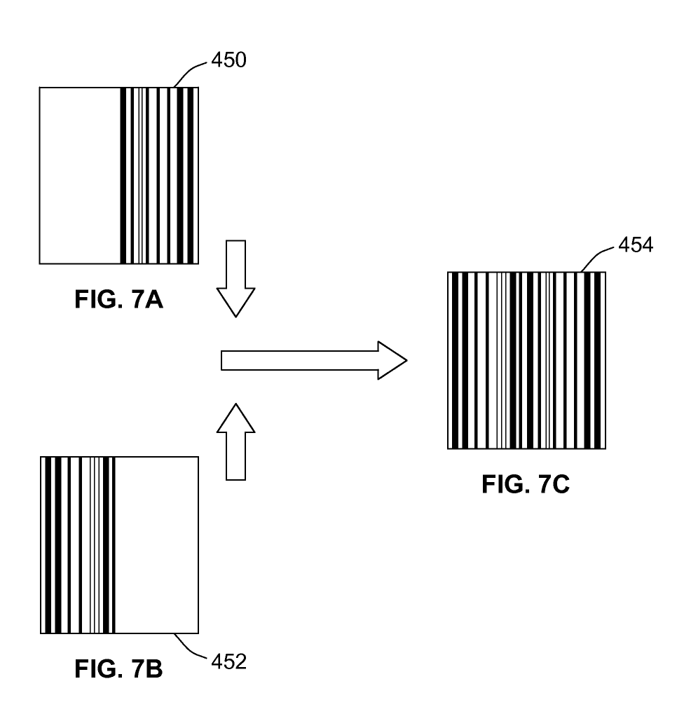

FIGS. 6-9 show additional examples of various code features that a coding system in accordance with the invention may include. FIGS. 6a, 6b, and 6c illustrate an example coding system where the visual indicator of the coding system is a drug logo. Two partial coding features 430, 432 that together form a drug logo 434. In an alternative example, the partial coding features may combine to form a bar code. FIGS. 7a, 7b, and 7c illustrate an example coding system where the visual indicator of the coding system is a bar code. First partial coding feature 450 includes a first portion of a bar code, and second partial coding feature 452 includes a second portion of the bar code. When the partial coding features are combined, they form a complete bar code 454.

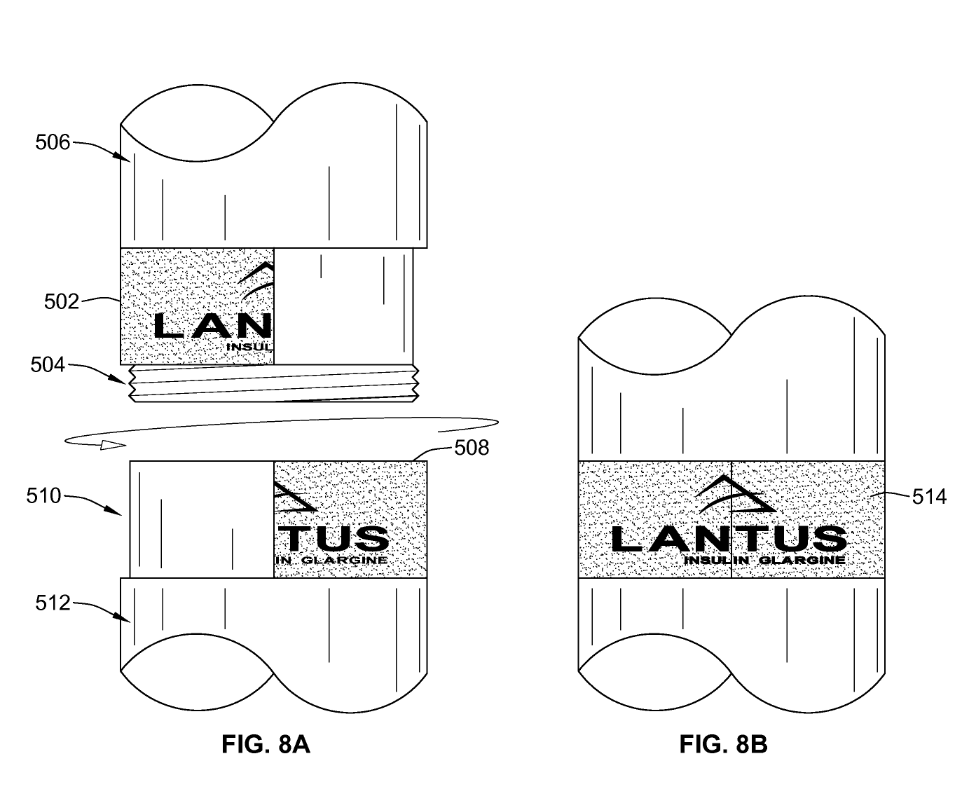

FIGS. 8a and 8b illustrate yet another example coding system for a drug delivery device. In this example, a first partial code feature 502 is located at a distal end 504 of dose setting mechanism 506. Further, a second partial code feature 508 is located at a proximal end 510 of holder 512. The distal end 504 is configured to threadably engage the proximal end 510. When the distal end 504 is engaged to the proximal end, a completed code 514 results.

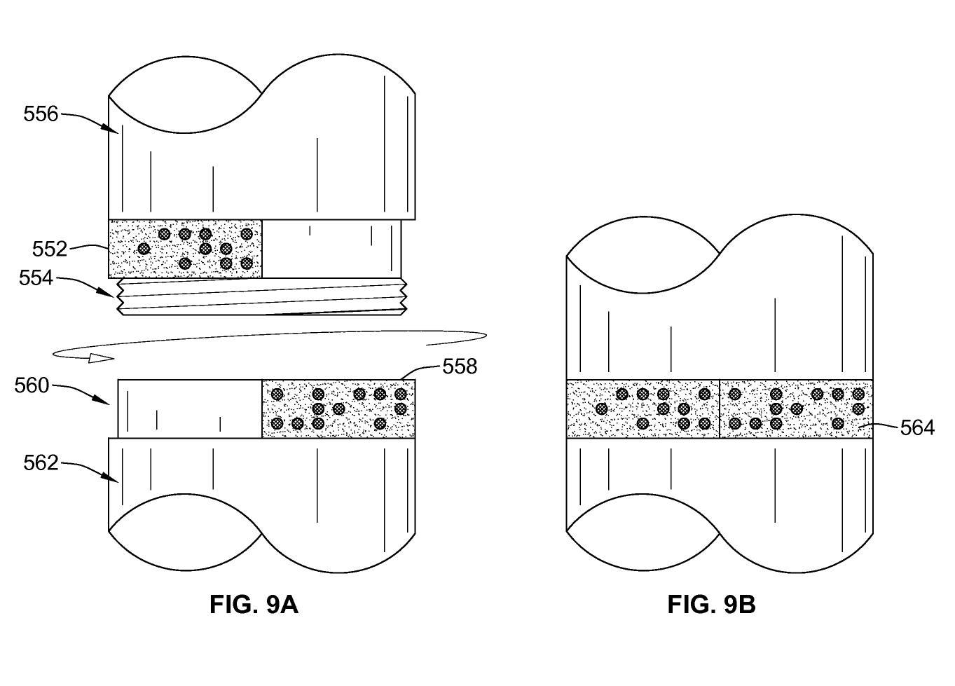

In another example, the partial coding features may combine to form a Braille code. This example may be of particular benefit to a user that is blind or has extreme difficulty seeing. FIGS. 9a and 9b illustrate an example coding system that forms a Braille code. In this example, a first partial code feature 552 is located at a distal end 554 of dose setting mechanism 556. Further, a second partial code feature 558 is located at a proximal end 560 of holder 562. The distal end 554 is configured to threadably engage the proximal end 560. When the distal end 554 is engaged to the proximal end, a completed Braille code 564 results. In this example, the completed Braille code reads “insulin”.

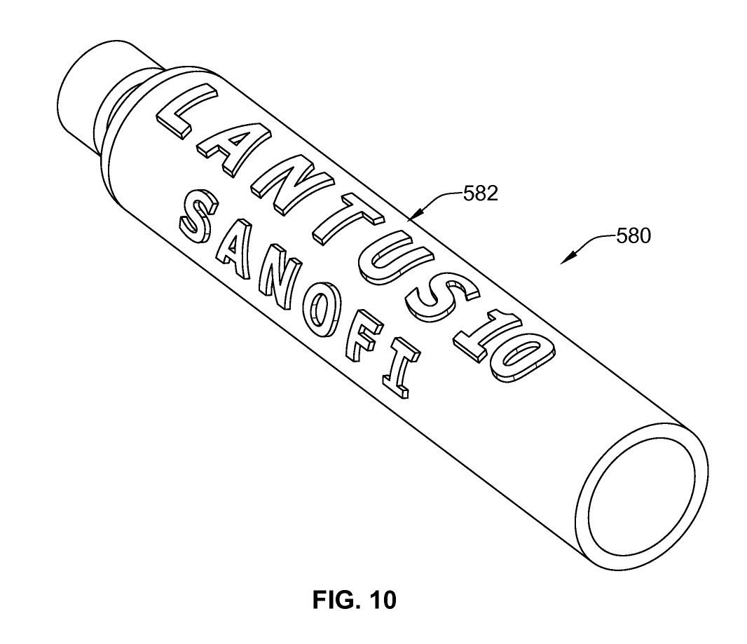

Another example coding system in accordance with the invention may include three dimensional coding features on a drug reservoir. An example cartridge 580 having a three-dimensional code 582 is shown in FIG. 10. The three dimensional coding features may be any desirable height including, for example, approximately 1-3 mm. In an example, the drug delivery system designed to dispense drug from a reservoir with a three-dimensional code will have corresponding indicia cut out of the reservoir retention component (e.g., holder). In the case of an injection pen, the cartridge holder would feature a set of cut-out indicia arrange to accommodate the raised indicia on cartridges containing the drug type for which the pen is designated. It is intended that reservoirs with the correct raised indicia will key with the reservoir holder cut-outs, and thus will allow the drug delivery device to assemble and operate correctly.

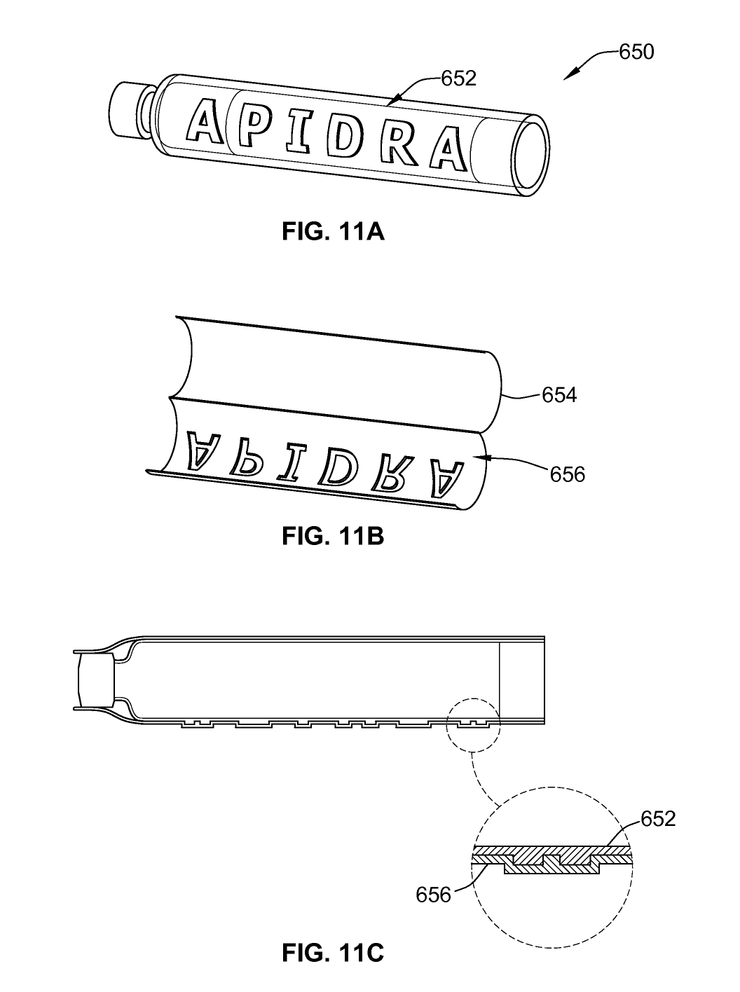

Example cut-outs are shown in FIG. 11. FIG. 11a shows a cartridge 650 having a three-dimensional code or raised indicia 652 spelling “APIDRA”. FIG. 11b shows a cartridge holder 654 intended for receiving cartridge 650. This holder 654 has corresponding cut-out 656, which also spells “APIDRA”. As shown in FIG. 11c, when the cartridge 650 and holder 654 are combined, the indicia 652 and cut-outs 656 line up with one another.

In another example coding system that includes a three-dimensional code feature, rather than complete cut-outs, the holder could comprise negative impressions. The negative impressions may be substantially the same shape as the three-dimensional feature and may have a depth that is approximately equal to the height of the three-dimensional features. In yet another example, the reservoir may comprise the negative impressions, while the reservoir holder comprises the raised indicia.

If a reservoir containing a given drug is inserted into an incorrect reservoir holder, the raised three-dimensional indicia will not match the cut-out indicia on the reservoir holder, and thus assembly and use of the drug delivery device will be prevented. Accordingly, reservoirs having a three-dimensional coding feature will typically only be able to be used with a correct holder.

In addition, a drug delivery device intended for use with a cartridge having a three-dimensional code may be designed to beneficially prevent a cartridge without a three-dimensional code from being used with the device. In order to accomplish this, the means with which the dispense interface (e.g., disposable needle) is connected to the drug delivery device is mounted on the cartridge itself. If a cartridge with the correct three-dimensional code is fitted into the drug delivery device, the indicia will prevent rotation of the reservoir as the dispense interface is screwed on. However, if a reservoir without any raised indicia is fitted in the drug delivery device, the reservoir will continue to rotate as a user attempts to screw on a dispense interface. This rotation will prevent the dispense interface from attaching, and therefore will prevent use of the drug delivery device with the incorrect cartridge. In other words, if the needle attachment is fixed to the reservoir, but not the reservoir holder, the needle attachment torque will have to be transmitted to the device via the reservoir. Without any anti-rotation features (i.e., the raised lettering), the reservoir will spin inside the reservoir holder, preventing attachment of the needle. Only a cartridge with the raised lettering will remain stationary during attachment of the needle, and thus allow use of the device.

In an alternative example of a coding system comprising three-dimensional features, the reservoir and the reservoir holder could each feature both three-dimensional coding and cut-out sections. This may beneficially increase the security of the coding system, making it even more difficult for an incorrect cartridge to be loaded.

Further, if the raised indicia are colored to contrast sharply with the drug delivery device, then the raised indicia will also help to provide differentiation features for the user to discriminate between cartridges and pen when a choice is presented to them.

Beneficially, a coding system having a three-dimensional code may provide enhanced drug delivery device and reservoir differentiation for users. The reservoir contents will be immediately visible, and the tactile quality of the raised letting may also help partially sighted users to identify the reservoir contents. If the coloring of the reservoir raised lettering is in strong contrast to the reservoir holder color, the users may easily be able to differentiate between drug delivery devices. Thus, the raised lettering may tie the drug delivery device to the drug reservoir both mechanically and visually.

In an example of the device, the drug delivery device may be configured to react to the combined information code once the first drug delivery system feature is attached to the second drug delivery system feature. For example, in the case where the combined information code is a bar code, the device may be configured to read the bar code. For example, a reservoir and reservoir holder may be combined to form a combined bar code, and a drug delivery device having a dose setting mechanism may read the bar code as the reservoir and reservoir holder are connected to the dose setting mechanism. In another bar code arrangement, the bar code could be used at different product phases to identify the correct assembly, during manufacturing, by a health care professional or pharmacist before the device is provided to the patient.

The device may additionally be configured to take an appropriate action if an incorrect reservoir and reservoir holder combination is loaded into a device. For instance, the drug delivery device may be configured to only allow loading of the reservoir into the device, dose selection, and/or dispensing of the drug if the combined bar code indicates that the reservoir and holder combination is intended for use with the device.

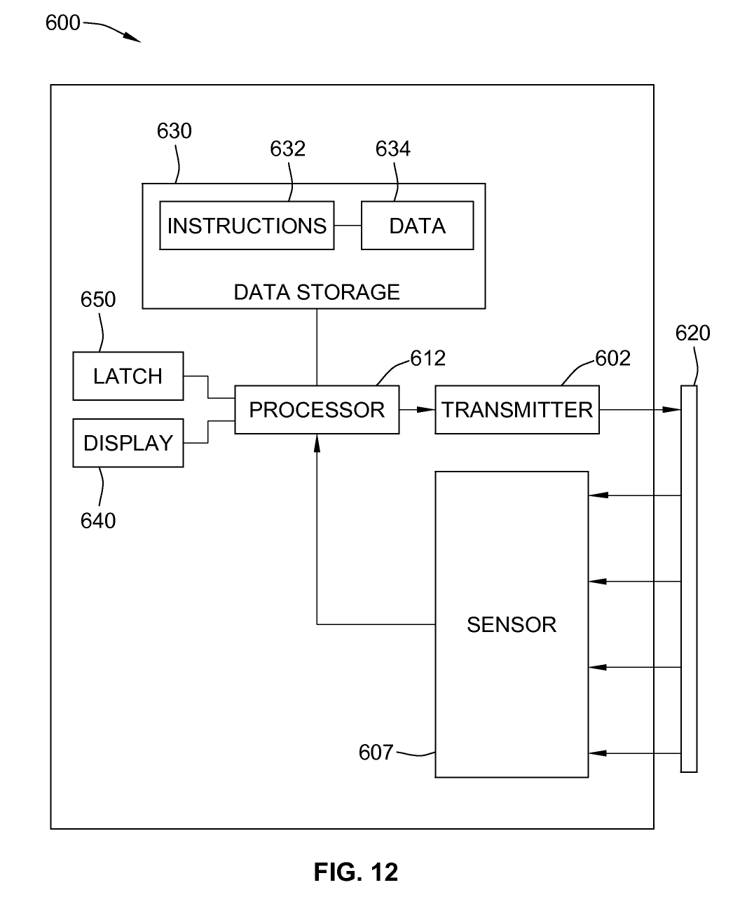

FIG. 12 depicts an example system 600 that may be included in a drug delivery device and that may read an information code (e.g., a bar code) and react to the determined information. System 600 includes transmitter 602, at least one receiver/sensor 607, and processor 612. The system may also comprise data storage 630 comprising instructions 632 executable by the processor 612 to carry out the functions described herein. The processor 612 may comprise a single processor such as a general purpose microprocessor or multiple (e.g., parallel) processors. The data storage 630 may take various forms, in one or more parts, such as a non-volatile storage block and/or a removable storage medium, and may include program instructions 632 executable by processor 612 for carrying out the system functions described herein. Data storage 630 may also include data 634, which may be used for carrying out the functions described herein.

The transmitter 602 and receiver/sensor 607 may act in conjunction with one another to read the combined bar code 620. After reading the bar code 620, the system 600 may determine if the bar code is a proper combined code (i.e., the drug delivery system features that are attached are intended for one another) or an incorrect combined code (i.e., the drug delivery system features that are attached are not intended for one another). The system 600 may then react to the identified information at various stages in an operating sequence of the drug delivery device. For instance, the system 600 may react to the information and take an appropriate action during (i) loading of the device, (ii) dose selection, and (iii) dispensing of the drug. Other stages are possible as well. Beneficially, during these steps, the system may help a user identify whether two combined drug delivery system features, such as a reservoir holder and a dose setting mechanism, are intended to be combined with one another and used with a given drug delivery device.

In an embodiment, when two drug delivery system features that are not intended to be combined with one another are inserted into a drug delivery device, the system may display an indication that the features are not intended for use with each other or with the drug delivery device. For instance, as shown in FIG. 12, the system 600 may comprise a display feature 640 that is in communication with the processor 612. This display feature 640 could indicate that the drug reservoir is not intended for use with the drug reservoir holder. For example, the display feature may display a red dot or red “X” when an incorrect combination of a reservoir and a holder is loaded into a device. The display feature 640 may also operate to indicate when a correct drug reservoir and reservoir holder combination is connected. For example, the display feature 640 may display a green dot when a correct drug reservoir holder is connected to the device. Other types of indications are possible as well, such as an audible indication.

If a user attempts to connect an incorrect reservoir and reservoir holder combination into the drug delivery device, the system 600 may operate to prevent the connection. For instance, preventing the connection of the drug reservoir holder and device may comprise activating an electronically-controlled latch, such as latch 650′ shown in FIG. 12, which prevents the connection of the drug reservoir holder.

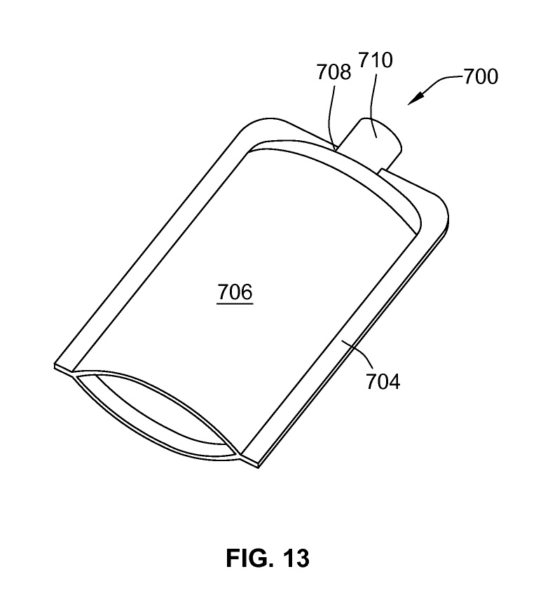

It should be understood that the proposed coding system may apply to any drug delivery device, with any type of reservoir or primary pack, e.g. inhaler, pouch. For example, FIG. 13 illustrates a drug reservoir 700 comprising a vessel 704 that contains a medicament 706. A stopper 708 is provided along a distal end of the vessel and is attached to the vessel so as to prevent the medicament 706 from exiting the vessel 704. The connector having the combined coding system described above may be provided on the output port 710 of the vessel. A partial code feature in accordance with the invention may, for example, be applied to the output port 710.

The proposed combined coding system results in a number of advantages. For example, the proposed combined coding mechanism assists a user to correctly match medicaments contained within a reservoir to a drug delivery device, thereby helping to indicate that the delivery device can only be used with a medicament for which the device is intended.

The dose setting mechanism also results in a low cost mechanism since the system does not require a large number of parts and can be manufactured in a cost effective manner. Moreover, there are quite a large number of different coding configurations between the cartridge and the cartridge holder that may be used. Consequently, with the proposed system, a large number of medicaments can be distinguished from one another. In addition, with the proposed system, if a user attempts to load an incorrect reservoir into a cartridge holder designed for a different cartridge, the user will be alerted at an early stage of the assembly process.

1. A coding system for use with a drug delivery device comprising a holder and a reservoir or cartridge sized and shaped to be received within the holder, the coding system comprising:
a first partial code feature; and
a second partial code feature, wherein the first partial code feature and the second partial code feature cooperate to supply a combined code feature when the reservoir is assembled to the holder for indicating that a correct reservoir has been assembled to the holder.

2. The coding system of claim 1, wherein the combined code feature comprises a visual indicator, wherein the visual indicator is one or more of a drug name, alphanumeric character, image, icon, symbol, design, trademark, word, phrase, number, bar code, or indicia.

3. The coding system of claim 1, wherein the combined code feature comprises a three-dimensional Braille code.

4. The coding system of claim 1, wherein the first partial code feature is disposed on an outside or inside surface of the reservoir.

5. The coding system of claim 1, wherein the first partial code feature is embedded within the material forming the reservoir.

6. The coding system of claim 1, wherein the first partial code feature includes one or more shaped areas, and the second partial code feature is one or more window formed through the holder, such that the one or more shaped areas can be viewed.

7. The coding system of claim 6, wherein the one or more shaped areas includes a plurality of elongate shapes, and the second partial code feature includes a number of windows formed through the holder corresponding in number, size and shape to the plurality of elongate shapes.

8. The coding system of claim 1, further comprising a third partial code feature, wherein the first partial code feature, the second partial code feature and the third partial code feature cooperate to supply a combined code feature when the reservoir is assembled to the holder.

9. The coding system of claim 1, further comprising an alignment feature, wherein the alignment feature aids the first partial code feature and the second partial code feature to cooperate so as to supply a combined code feature when the reservoir is assembled to the holder for indicating that a correct reservoir has been assembled to the holder.

10. The coding system of claim 1, further comprising a protrusion, wherein the protrusion is configured to mesh with either the reservoir holder or the reservoir so as to align the first and second partial codes.

11. The coding system of claim 1, wherein the holder is mechanically coded to a dose setting mechanism.

12. The coding system of claim 1, further comprising:
a drug reservoir comprising a three-dimensional coding feature as a first partial code feature disposed on an external portion of the outer body; and a corresponding holder or receiving the drug reservoir, the holder comprising a corresponding coding feature as a second partial code feature for receiving the three-dimensional coding feature.

13. The coding system of claim 12, wherein the corresponding coding feature is a negative impression in an inner surface of the holder, wherein the negative impression is substantially the same shape as the three-dimensional coding feature.

14. The coding system of claim 12, wherein the corresponding coding feature is a cut-out that is substantially the same shape as the three-dimensional coding feature.

15. A drug delivery system, the system comprising:
a drug delivery device comprising a dose setting mechanism and a cartridge holder removably coupled to the dose setting mechanism;
a cartridge sized and shaped to be contained within the cartridge holder and operably responsive to the dose setting mechanism; and
a coding system for use with the drug delivery device including a first partial code feature;
a second partial code feature, wherein the first partial code feature and the second partial code feature cooperate to supply a combined visual indicator when the cartridge is assembled to the cartridge holder for indicating that a correct reservoir has been assembled to the holder.

16. The drug delivery system of claim 15, wherein the drug delivery system comprises a reusable drug delivery system.

17. The drug delivery system of claim 15, wherein the drug delivery system comprises a non-reusable drug delivery system.

18. A coding system for use with a drug delivery system, the coding system comprising:
a first partial code feature disposed on a first drug delivery system feature; and a second partial code feature disposed on a second drug delivery system feature, wherein the first drug delivery system feature is at least in principle connectable to the second drug delivery system features, wherein the first partial code feature and the second partial code feature cooperate to supply a combined information code when the first drug delivery system feature is connected to the second drug delivery system feature for indicating that a correct second drug delivery system feature has been connected to the first drug delivery system feature.
